(19) United States
(12) Patent Application Publication
(10) Pub. No.: US 2007/0060489 A1
(43) Pub. Date: Mar. 15, 2007

(54) Cationic surfactant shampoo composition

(76) Inventors: James Ziming Sun, Costa Mesa, CA (US); Thomas Foerster, Erkrath (DE); Erik Schulze zur Wiesche, Hamburg (DE); Sabine Albrechtsen, Elmshorn (DE)

Correspondence Address: PAUL & PAUL, 2000 MARKET STREET, PHILADELPHIA, PA, 19103-3229, US

(21) Appl. No.: 11/355,869

(22) Filed: Feb. 16, 2006

Related U.S. Application Data
(60) Provisional application No. 60/653,732, filed on Feb. 17, 2005.

(51) Int. Cl. A61K 8/00

(52) U.S. Cl. 510/119; 8/405

(57) ABSTRACT

A cleaning agent for keratinic fibers which is essentially free of anionic surfactants is comprised, based on the weight of the cleaning agent, of from 1 to 50 wt. % of one or more cationic surfactants of 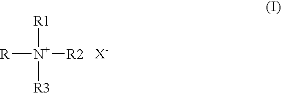
wherein R is an alkyl group having from 8 to 18 carbon atoms; each of R¹, R², and R³ is independently a C₁-C₃-alkyl group and X⁻ is a halogen or methosulfate. The cleaning agents can be used for conditioning and color protection of hair as well as the reduction and prevention of damage to hair.

### CROSS REFERENCE TO RELATED APPLICATIONS

This application claims the benefit of copending Provisional Application Ser. No. 60/653,732, filed on Feb. 17, 2005, the entire contents of which are incorporated herein by reference.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The application relates to essentially anionic surfactant-free cleaning agents for keratinic fibers based on specific cationic surfactants and the use of the cleaning agent for conditioning and color protection of hair as well as the reduction and prevention of damage to hair.

Nowadays, human hair of the head is treated with a variety of cosmetic preparations that are intended to make the hair glossy and easily combable, and lend the hair a healthy external appearance. The variety of treatments of hair, for example, bleaching, dyeing, tinting and setting, can however cause unwanted damage to the hair texture. Damage to the hair texture is noticeable, for example, by a poor wet and dry combability, an increased electrostatic charge, increased brittleness, reduced maximum tear force and elongation at break of the hair, split ends and an overall worsened external appearance of the hair.

There is therefore a need for hair cleaning agents that simultaneously clean and care for hair, without the need for an additional hair treatment step with a special hair care product. Due to the increased sales of hair dyes that damage the hair texture more and more, particularly on repeated hair dyeing, hair cleaning agents are strived for which prevent hair damage from dyeing or bleaching. Cleaning with these agents should however not lead to any discoloration, but should rather simultaneously maintain a color protection.

The objective was therefore to fulfill all these demands in one product.

A lot of effort has been spent in the last 40 years to improve the conditioning performance of shampoos, in that cationic polymers and/or silicones were incorporated as hair conditioner components in anionic surfactant systems.

By themselves, cationic polymers do not provide a satisfactory conditioning effect, particularly concerning the suppleness and the wet and dry combability of hair.

On the other hand, insoluble, non-volatile silicone oils are widely used as good conditioners in hair-care agents, but often leave the hair with a poor, heavy and greasy touch.

(2) Description of Related Art, Including Information Disclosed Under 37 C.F.R. §§ 1.97 and 1.98

For these reasons, many experiments were carried out in the prior art with the aim of combining both these components in hair-care and cleaning agents so as to add the positive effects and to largely eliminate their disadvantages. For example, in EP 892 631 B1 conditioning shampoos were proposed, which include a combination of special cationic polymers, insoluble silicones and special surfactants. Similar combinations are known from the documents WO 92/10162 A1, WO 94/031515 A1 and WO 95/22311 A1.

The bases for conditioning shampoos of the prior art are largely the same: an anionic surfactant system, the cationic polymer and silicones were blended so as to also obtain a conditioning effect at the same time as the cleaning.

The use of cationic surfactants as cationic conditioning components in anionic surfactant systems is also known from several hair cleaning and care products. In particular, cationic surfactants with a chain length of 16 to 22 carbon atoms were added for this purpose. As a result of their cationic character, they are able to neutralize the anionic hair surfaces and therefore to reduce the static charge of hair. Compared with anionic surfactants, which are normally used in hair cleaning agents, these surfactants have a low foaming ability, however, which is the reason why previously their addition was largely limited to hair conditioners and hair cures. They were also added in addition to anionic and/or amphoteric surfactants in hair cleaning agents at best in low concentrations.

Completely surprisingly, it has now been found that cationic surfactants with a chain length of 8 to 18 carbon atoms posses a sufficient foamability and cleaning capability for use as the cleaning surfactant in a hair-cleaning agent. At the same time, they offer the advantage that they are gentle to the hair and the scalp, do not leave any visual film on the hair, improve the wet and dry combability, leave a pleasant feel to the scalp and are toxicologically harmless. Moreover, shampoos based on these surfactants are able to prevent damage to hair and lend a color protection to dyed hair.

In addition, they permit the incorporation of polyethylene imine (PEI), the most effective active principle for healing and prevention of damage to hair. On the contrary, PEI cannot be incorporated into normal shampoos.

### BRIEF SUMMARY OF THE INVENTION

Accordingly, the subject of the invention is cleaning agents for keratinic fibers, comprising—based on their weight—1 to 50 wt. % of one or more cationic surfactants of Formula (I) 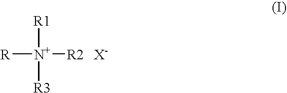
wherein
R stands for an alkyl group with 8 to 18 carbon atoms,
R¹, R², R³ stand independently of one another for a C₁-C₃-alkyl group and
X⁻ stands for halogen or methosulfate, and are essentially free of anionic surfactants.

### BRIEF DESCRIPTION OF THE SEVERAL VIEWS OF THE DRAWING(S)

Not Applicable

### DETAILED DESCRIPTION OF THE INVENTION

According to the invention, the term, “essentially free of anionic surfactants,” means that the content of anionic surfactants in the cleaning agents is <5 wt. %, preferably <3 wt. % and particularly <1.5 wt. %.

According to the invention, suitable cationic surfactants are those in which the group R stands for an alkyl group with 8 to 18 carbon atoms and preferably for an alkyl group with 10 to 14 carbon atoms.

Particularly suitable cationic surfactants in the context of the invention are Cocotrimonium Chloride, Cocotrimonium Methosulfate, Laurtrimonium Chloride and/or Laurtrimonium Methosulfate.

The cationic surfactants are added in the inventive cleaning agents—based on their weight—in an amount from 1 to 50 wt. % and particularly in an amount from 5 to 30 wt. %.

Cleaning agents can further comprise—based on its weight—0.1 to 5 wt. %, particularly 0,3 to 3 wt. % of one or more foam strengtheners from the group of the non-ionic and/or the amphoteric surfactants.

In a preferred embodiment of the invention, the cleaning agents comprise a foam strengthener from the group of the fatty acid alkanolamides, the betaines, the amine oxides and/or the sugar surfactants.

Inventively suitable betaines include, for example, cocoalkyldimethylammonium glycinate, N-acyl-aminopropyl-N,N-dimethylammonium glycinate and 2-alkyl-3-carboxymethyl-3-hydroxyethylimidazoline, each having 8 to 18 carbon atoms in the alkyl or acyl group, as well as cocoacylaminoethylhydroxyethylcarboxymethyl glycinate. A particularly preferred zwitterionic surfactant is the fatty acid amide derivative, known under the INCl name cocoamidopropyl betaine.

Inventively suitable fatty acid alkanolamides include those with fatty acid chains of 8 to 30 carbon atoms. According to the invention, addition products of ethylene oxide on fatty acid alkanolamides and fatty amines also fall in this category.

Inventively suitable sugar surfactants are understood to mean alkyl polyglycosides corresponding to the general formula RO—(Z)ₓ, wherein R stands for alkyl, Z for sugar and x for the number of sugar units. The alkyl polyglycosides used according to the invention may simply comprise a defined alkyl group R. However, normally these compounds are manufactured from natural fats and oils or mineral oils. In which case, the alkyl groups R are present as mixtures corresponding to the starting compounds or to each of the compounds worked up.

Such alkyl polyglycosides are particularly preferred in which R consists
essentially of C₈- and C₁₀ alkyl groups,
essentially of C₁₂- and C₁₄ alkyl groups,
essentially of C₈ to C₁₆ alkyl groups or

1essentially of C₁₂- to C₁₆ alkyl groups or
essentially of C₁₆- to C₁₈ alkyl groups.

Any mono or oligosaccharide can be added as the sugar building block Z. Usually, sugars with 5 or 6 carbon atoms as well as the corresponding oligosaccharides are used. Such sugars are, for example, glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred.

The alkyl polyglycosides used according to the invention comprise on average 1.1 to 5 sugar units. Alkyl polyglycosides with x-values of 1.1 to 2.0 are preferred. Alkyl polyglycosides with x-values of 1.1 to 1,8 are quite particularly preferred.

The alkoxylated homologs of the cited alkyl polyglycosides can also be used according to the invention. These homologs can comprise on average up to 10 ethylene oxide and/or propylene oxide units per alkyl glycoside unit.

Particularly suitable foam strengtheners in the context of the invention are Cocamide MEA, Cocamide DEA, Cocamide MIPA, PEG-2 Cocamide, PPG-2 Hydroxyethyl Cocamide, PPG-2 Hydroxyethyl Coco/Isostearamide, alkylamidopropyl betaines and/or alkylamidopropylamine oxides.

In a particularly preferred embodiment of the invention, the cleaning agents further comprise 0.5 to 2 wt. %, particularly 0.1 to 1 wt. % of a polyalkylene imine for healing and preventing damage to the hair. According to the invention, a particularly suitable polyalkylene imine is polyethylene imine (PEI).

Normally, the inventive cleaning agents comprise a thickener. Thickeners that are added in anion surfactant-based shampoos are unsuitable for the inventive cationic shampoos, as they exhibit no stable thickening effect in them.

According to the invention, hydroxypropyl guar derivatives and/or hydroxyethyl ethylcellulose therefore serve as thickeners and are added to the cleaning agents in a quantity of 0.1 to 3 wt. %, preferably in a quantity of 0.3 to 2 wt. %.

According to the invention, particularly suitable thickeners are the hydroxypropyl guar derivatives, commercialized under the trade names Jaguar HP-8, Jaguar HP-60, Jaguar HP-105, Jaguar HP-120, Jaguar HP-200, N-Hance HP-40, N-Hance HP-40S as well as hydroxyethyl ethylcellulose derivatives obtainable commercially under the trade names Elfacos CD-481 and Elfacos CDHM.

In a further preferred embodiment of the invention, the cleaning agents comprise—based on their weight—in addition 0.05 to 3 wt. %, particularly 0.1 to 2 wt. % of an additional cationic surfactant, selected from alkyltrimethylammonium chlorides or—methosulfates, dialkyldimethylammonium chlorides or—methosulfates and/or trialkylmethylammonium chlorides or—methosulfates with an alkyl chain length of 16 to 22 carbon atoms. According to the invention, particularly preferred cationic surfactants of this group are Behentrimonium chloride and Cetrimonium methosulfate.

As a further preferred embodiment, the inventive cleaning agents—based on their weight —0.05 to 2 wt. %, particularly 0.1 to 1 wt. % of at least one styling polymer. Preferably, they are selected from the group Polyquaternium-55, Polyquaternium-46, Polyquaternium-11 and/or Polyquaternium-28.

Quite particularly preferred are inventive cleaning agents that—based on their weight —comprise in addition 0.05 to 3 wt. %, particularly 0.1 to 2 wt. % of a water-soluble or water-insoluble, volatile or non-volatile silicone component.

Inventive agents are especially preferred that comprise a silicone, selected from:
(i) volatile or non-volatile, linear or cyclic, crosslinked or non-crosslinked polyalkylsiloxanes, polyarylsiloxanes, polyalkylarylsiloxanes;
(ii) polysiloxanes, which comprise one or more organofunctional groups in their general structure, selected from:
   substituted or unsubstituted aminated groups;
a) (per)fluorinated groups;
b) thiol groups;
c) carboxylate group
d) hydroxylated groups;
e) alkoxylated groups;
f) acyloxyalkyl groups;
g) amphoteric groups;
h) bisulfite groups;
i) hydroxyacylamino groups;
j) carboxy groups;
k) sulfonic acid groups; and
l) sulfate or thiosulfate groups;
(iii) linear polysiloxane(A)-polyoxyalkylene(B) block copolymers of the type (A-B)ₙ with n>3;
(iv) grafted silicon polymers with non silicon-containing organic structures that consist of an organic backbone that is formed from organic monomers that do not comprise silicon, on which in the chain as well as optionally on at least one chain end at least one polysiloxane macromer has been grafted;
(v) grafted silicon polymers with polysiloxane backbone, grafted onto the non silicon-containing organic monomer, which possess a polysiloxane main chain on which in the chain as well as optionally on at least one chain end at least one organic macromer has been grafted that comprises no silicon;
(vi) or their mixtures.

Particularly preferred inventive hair treatment agents are thus wherein they comprise at least one silicone of Formula (I)
(CH₃)₃Si—[O—Si(CH₃)₂]ₓ—O—Si(CH₃)₃ (I),
in which x stands for a number from 0 to 100, advantageously from 0 to 50, more preferably from 0 to 20 and especially 0 to 10.

The inventively preferred hair treatment agents comprise a silicone of the above-mentioned Formula I. These silicones are designated according to the INCl nomenclature as DIMETHICONES.

According to the invention, particularly preferred dimethicones are those that have a viscosity at 20° C.>6000 cSt, preferably >20 000 cSt and particularly >40 000 cSt.

Particularly preferred agents according to the invention comprise one or more aminofunctional silicones. Such silicones can be described by the Formula
M(RₐQ_{b}SiO_{(4-a-b)/2})ₓ(R_{c}SiO_{(4-c/2})_{y}M
wherein, in the above formula R is a hydrocarbon or a hydrocarbon group with 1 to 6 carbon atoms, Q is a polar group of the general formula —R¹HZ, wherein R¹ is a divalent, linking group that is bonded to hydrogen and the group Z, made up of carbon atoms and hydrogen atoms, carbon-, hydrogen- and oxygen atoms or carbon-, hydrogen- and nitrogen atoms, and Z is an organic amino functionalized group that comprises at least one aminofunctional group; “a” assumes values in the range of about 0 to about 2, “b” assumes values in the range of about 1 to about 3, “a”+“b” is less than or equal to 3, and “c” is a number in the range of about 1 to about 3, and x is a number in the range of 1 to about 2000, advantageously from about 3 to about 50 and most preferably from about 3 to about 25, and y is a number in the range of about 20 to about 10 000, advantageously from about 125 to about 10 000 and most preferably from about 150 to about 1000, and M is a suitable silicone end-group, as is known from the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl and the like; alkenyl groups, such as vinyl, halogenovinyl, alkylvinyl, allyl, halogenoallyl, alkylallyl; cycloalkyl groups, such as cyclobutyl, cyclopentyl, cyclohexyl and the like; phenyl groups, benzyl groups, halogenated hydrocarbon groups, such as 3-chloropropyl, 4-bromobutyl, 3,3,3-trifluoropropyl, chlorocyclohexyl, bromophenyl, chlorophenyl and the like as well as sulfur-containing groups, such as mercaptoethyl, mercaptopropyl, mercaptohexyl, mercaptophenyl and the like; advantageously R is an alkyl group that comprises 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂₃C(O)SCH₂CH₂—.

Z is an organic, aminofunctional group comprising at least one functional amino group. A possible formula for Z is NH(CH₂)_{z}NH₂, wherein z is 1 or more. Another possible formula for Z is —NH(CH₂)_{z}(CH₂)_{zz}NH, wherein both z and zz independently are 1 or more, wherein this structure includes diamino ring structures, such as piperazinyl. Most preferably, Z is an —NHCH₂CH₂NH₂ group. Another possible formula for Z is —N(CH₂)_{z}(CH₂)_{zz}NX₂ or —NX₂, in which each X of X₂ is independently selected from the group consisting of hydrogen and alkyl groups with 1 to 12 carbon atoms, and zz is 0.

Most preferably, Q is a polar, amine functional group of formula —CH₂CH₂CH₂NHCH₂CH₂NH₂. In the formulas “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b} SiO_{(4-a-b)1/2} units to the R_{c}SiO_{(4-c)1/2} units is in the range from about 1:2 to 1:65, preferably from about 1:5 to about 1:65 and most preferably from about 1:15 to about 1:20. If one or a plurality of silicones of the above formula are added, then the different variable substituents in the above formula for the different silicone components that are present in the silicone mixture can be different.

Preferred inventive hair treatment agents are characterized in that they comprise an aminofunctional silicone of Formula (II)
R′ₐG₃₋ₐ—Si(OSiG₂)ₙ—(OSiG_{b}R′_{2-b})ₘ—SiG₃₋ₐ—R′ₐ (II),
wherein:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;

a stands for a number between 0 and 3, particularly 0;

b stands for a number between 0 and 1, particularly 1;

m and n are numbers whose sum (m+n) is between 1 and 2000, preferably between 50 and 150, wherein n preferably assumes values of 0 to 1999 and particularly 49 to 149 and m preferably assumes values of 1 to 2000, particularly 1 to 10,

R is a monovalent group selected from
Q-N(R″)—CH₂—CH₂—N(R″)₂
-Q-N(R″)₂
-Q-N⁺(R″)₃A⁻
-Q-N⁺H(R″)₂A⁻
-Q-N⁺H₂(R″)A⁻
-Q-N(R″)—CH₂—CH₂—N⁺R″H₂A⁻,
   wherein each Q stands for a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,

R″ stands for the same or different groups from the group —H, -phenyl, -benzyl, —CH₂—CH(CH₃)Ph, the C₁₋₂₀-alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and

A represents an anion that is preferably selected from chloride, bromide, iodide or methosulfate.

Particularly preferred inventive hair treatment agents are characterized in that they comprise at least one aminofunctional silicone of Formula (IIa) 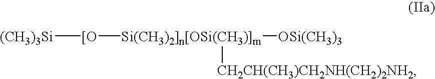
in which m and n are numbers whose sum (m+n) is between 1 and 2000, preferably between 50 and 150, wherein n preferably assumes values of 0 to 1999 and particularly 49 to 149 and m preferably assumes values of 1 to 2000, particularly 1 to 10,

These silicones are designated according to the INCl nomenclature as Trimethylsilylamodimethicones.

Particularly preferred inventive hair treatment agents are also those that comprise at least one aminofunctional silicone of Formula (IIb) 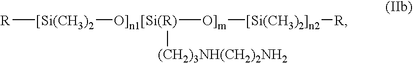
in which R stands for —OH, —O—CH₃ or a —CH₃ group and m, n1 and n2 are numbers whose sum (m+n1+n2) is between 1 to 2000, preferably between 50 and 150, wherein the sum (n1+n2) preferably assumes values of 0 to 1999 and particularly 49 to 149 and m preferably assumes values of 1 to 2000, particularly 1 to 10.

These silicones are designated according to the INCl nomenclature as Amodimethicones.

Independently of which aminofunctional silicone is added, inventive hair treatment agents are preferred that comprise an aminofunctional silicone whose amino number is above 0.25 meq/g, preferably above 0.3 meq/g and particularly above 0.4 meq/g. The amine number stands for the milliequivalents of amine per gram of aminofunctional silicone. It can be measured by titration and is also reported with the unit mg KOH/g.

According to the invention, preferred hair treatment agents are characterized in that they comprise, based on their weight, 0.01 to 10 wt. %, preferably 0.1 to 8 wt. %, particularly preferably 0.25 to 7.5 wt. % and particularly 0.5 to 5 wt. % aminofunctional silicone(s).

Also, according to the invention, the addition of cyclic dimethicones, designated by INCl as CYCLOMETHICONES, is preferred. Here, inventive hair treatment agents are preferred that comprise at least one silicone of Formula III 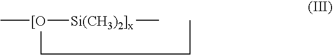
in which x stands for a number from 0 to 200, advantageously from 0 to 10, more preferably from 0 to 7 and especially 0, 1, 2, 3, 4, 5 or 6.

The above-described silicones possess a backbone that is constructed from —Si—O—Si— units. Of course, these Si—O—Si units can also be interrupted by carbon chains. Appropriate molecules are obtained by chain extension reactions and are preferably added in the form of silicone in water emulsions.

The silicone in water emulsions that are added according to the invention can be prepared by means of known processes, as disclosed, for example, in U.S. Pat. No. 5,998,537 and EP 0 874 017 A1.

In summary, this manufacturing process includes the emulsifiable mixture of components, one comprising at least one polysiloxane, the other comprising at least one organosilicon material that reacts with the polysiloxane in a chain extension reaction, wherein at least one chain extension reaction catalyst that contains a metal ion is present, as well as a surfactant and water.

Chain extension reactions with polysiloxanes are known and can include, for example, the hydrosilylation reaction, in which a Si—H group is reacted with an aliphatic unsaturated group in the presence of a platinum/rhodium catalyst to form polysiloxanes with several Si—(C)ₚ—Si bonds (p=1-6), the polysiloxanes being also designated as polysiloxane-polysilalkylene copolymers.

The chain extension reaction can also include the reaction of an Si—OH group (for example, a hydroxyl terminated polysiloxane) with an alkoxy group (for example, alkoxy silanes, silicates or alkoxysiloxanes) in the presence of a metal-containing catalyst to afford polysiloxanes.

The polysiloxanes that are used in the chain extension reaction include a substantially linear polymer of the following structure:
R—Si(R₂)—[—O—Si(R₂)—]ₙ—O—SiR₃

In this structure, each R independently of each other stands for a hydrocarbon group with up to 20 carbon atoms, preferably with 1 to 6 carbon atoms, such as, for example, an alkyl group (for example, methyl, ethyl, propyl or butyl), an aryl group (for example phenyl), or the group required for the chain extension reaction (“reactive group,” for example, Si-bonded hydrogen atoms, aliphatic unsaturated groups like vinyl, allyl or hexenyl, hydroxy, alkoxy like methoxy, ethoxy or propoxy, alkoxy-alkoxy, acetoxy, amino etc.) with the proviso that on average, one or two reactive groups per polymer are present, n is a positive number >1. Preferably, an excess of reactive groups, particularly preferably >90%, and in particular >98% of the reactive groups is bonded to the terminal Si atom in the siloxane. Preferably, n stands for numbers that describe what viscosities between 1 and 1 000 000 mm²/s the polysiloxanes have, particularly preferably viscosities between 1000 and 100 000 mm²/s.

The polysiloxanes can be branched to a small extent (for example, <2 mol % of the siloxane units), or the polymers are substantially linear, particularly preferably completely linear. In addition, the substituents R can themselves be substituted, for example, by N-containing groups (for example, amino groups), epoxy groups, S-containing groups, Si-containing groups, 0-containing groups etc. Preferably, at least 80% of the R groups are alkyl groups, particularly preferably methyl groups.

The organosilicon material that reacts with the polysiloxane in the chain extension reaction can either be a second polysiloxane, or a molecule that acts as a chain extender. If the organosilicon material is a polysiloxane, then it has the above-mentioned general structure. In these cases one polysiloxane possesses (at least) one reactive group in the reaction, and a second polysiloxane possesses (at least) one second reactive group that reacts with the first.

When the organosilicon material includes a chain extender, then this can be a material such as, for example, a silane, a siloxane (for example, disiloxane or trisiloxane) or a silazane. Thus, for example, a composition that includes a polysiloxane according to the above-described general structure, which possesses at least one Si—OH group, can be chain extended by its reaction with an alkoxysilane (for example, dialkoxysilane or trialkoxysilane) in the presence of a tin- or titanium containing catalyst.

The metal-containing catalysts in the chain extension reaction are mostly specific for a particular reaction. Such catalysts are known from the prior art and comprise, for example, metals like platinum, rhodium, tin, titanium, copper, lead, etc. In a preferred chain extension reaction, a polysiloxane having at least one aliphatic unsaturated group, preferably an end group, is reacted in the presence of a hydrosilylation catalyst with an organosilicon material that is a siloxane or polysiloxane having at least one (preferably terminal) Si—H group. The polysiloxane possesses at least one aliphatic unsaturated group and satisfies the above-mentioned general formula, in which R and n are as previously defined, wherein on average, between 1 and 2 R groups per polymer possess an aliphatic unsaturated group. Representative aliphatic unsaturated groups are, for example, vinyl, allyl, hexenyl and cyclohexenyl or a group R²CH═CHR³, in which R² stands for a divalent aliphatic chain linked to the silicon and R³ stands for a hydrogen atom or an alkyl group. The organosilicon material having at least one Si—H group has preferably the above cited structure, in which R and n are as previously defined, wherein on average, between 1 and 2 R groups mean a hydrogen and n is 0 or a positive number.

This material can be a polymer or a low molecular weight material like a siloxane (for example, a disiloxane or a trisiloxane).

The polysiloxane, having at least one aliphatic unsaturated group, and the organosilicon group, having at least one Si—H group, react in the presence of a hydrosilylation catalyst. Such catalysts are known from the prior art and include, for example, platinum- and rhodium-containing materials. The catalysts can be in any known form, for example, platinum or rhodium deposited on carrier materials (for example, silica gel or active charcoal) or other suitable compounds like platinum chloride, salts of platinic acid or chloroplatinic acids. Due to its good dispersability in organosilicon systems and to the low color changes, a preferred catalyst is chloroplatinic acid, either as the commercially available hexahydrate or in anhydrous form.

In a further preferred chain extension reaction, a polysiloxane having at least one Si—OH group, preferably an end group, is reacted with an organosilicon material that has at least one alkoxy group, preferably a siloxane having at least one Si—OR group or an alkoxysilane having at least two alkoxy groups. Again, a metal-containing catalyst is again used as the catalyst here.

For the reaction between an Si—OH group with a Si—OR group, there exist many catalysts known from the literature, for example, organometallic compounds like organotin salts, titanates or titanium chelates or complexes. Examples include tin octoate, dibutyltin dilaurate, dibutyltin diacetate, dimethyltin dineodecanoate, dibutyltin dimethoxide, isobutyltin triceroate, dimethyltin dibutyrate, dimethyltin dineodecanoate, triethyltin tartrate, tin oleate, tin naphthenate, tin butyrate, tin acetate, tin benzoate, tin sebacate, tin succinate, tetrabutyltitanate, tetraisopropyltitanate, tetraphenyltitanate, tetraoctadecyltitanate, titanium naphthanate, ethyltriethanolamine titanate, titanium diisopropyl diethyl acetoacetate, titanium diisopropoxy diacetyl acetonate and titanium tetra alkoxide, in which the alkoxide is butoxy or propoxy.

Furthermore, the silicone in water emulsions preferably comprise at least one surfactant. They were described in detail above.

Likewise preferred inventive hair treatment agents are thus characterized in that they comprise at least one silicone of Formula (IV)
R₃Si—[O—SiR₂]ₓ—(CH₂)ₙ—[O—SiR₂]_{y}—O—SiR₃ (IV),
in which R stands for the same or different groups —H, -phenyl, -benzyl, —CH₂—CH(CH₃)Ph, the C₁₋₂₀-alkyl groups, preferably —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, x or y stands for a number from 0 to 200, advantageously from 0 to 10, more preferably from 0 to 7 and especially 0, 1, 2, 3, 4, 5 or 6, and n stands for a number from 0 to 10, preferably from 1 to 8 and particularly for 2, 3, 4, 5, 6.

Preferably, the silicones of the inventive cleaning agent are water-insoluble. According to the invention, preferred hair treatment agents are thus characterized in that they may further comprise a water-insoluble silicone.

The inventive shampoos may, in addition, comprise vitamins, protein hydrolyzates, polyols, cationic and/or non-ionic polymers, plant extracts, pearlizing agents, opacifiers, perfume components, pH regulators, dyestuffs, conservation agents, optional fats as well as viscosity regulators.

Exemplary pearlizing waxes include: alkylene glycol esters; fatty acid alkanolamides; partial glycerides; esters of polyfunctional, optionally hydroxy substituted carboxylic acids with fatty alcohols with 6 to 22 carbon atoms; fats, such as, for example, fatty alcohols, fatty ketones, fatty aldehydes, fatty ethers and fatty carbonates, which have a total of at least 24 carbon atoms; ring opened products of olefin epoxides with 12 to 22 carbon atoms with fatty alcohols with 12 to 22 carbon atoms and/or polyols with 2 to 15 carbon atoms and 2 to 10 hydroxyl groups as well as mixtures thereof.

In a further preferred embodiment of the invention, fats can be comprised in addition. Fats are understood to mean fatty acids, fatty alcohols, natural and synthetic waxes that can exist both in solid form as well as liquid in aqueous dispersion, and natural and synthetic cosmetic oil components.

Linear and/or branched, saturated and/or unsaturated fatty acids having 6-30 carbon atoms can be used as the fatty acids. Fatty acids having 10-22 carbon atoms are preferred. Among these may be cited the isostearic acids, such as the commercial products Emersol®871 and Emersol®875, and isopalmitic acids such as the commercial product Edenor® IP 95, as well as all other fatty acids commercialized under the trade names Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linolic acid, linolenic acid, elaeostearic acid, arachidonic acid, gadoleic acid, behenic acid and erucic acid as well as their technical mixtures, that result, for example, from cracking of natural fats and oils, from the oxidation of aldehydes from Roelen's oxo synthesis or the dimerization of unsaturated fatty acids. Usually, the fatty acid fractions obtainable from coconut oil and palm oil are particularly preferred; in general, the addition of stearic acid is particularly preferred.

The addition quantity ranges from 0.1-15 wt. %, based on the total agent. In a preferred embodiment, the quantity ranges from 0.5-10 wt. %, quantities of 1-5 wt. % being quite particularly advantageous.

As fatty alcohols, saturated, mono or polyunsaturated, branched or linear fatty alcohols with C₆ to C₃₀-, preferably C₁₀ to C₂₂- and quite particularly preferably C₁₂ to C₂₂- carbon atoms can be added. In the scope of the invention, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, caprinic alcohol, linoleyl alcohol, linolenyl alcohol and behenyl alcohol, as well as the Guerbet alcohols can be added, this listing being intended as exemplary and not limiting. However, the fatty alcohols are preferably derived from naturally occurring fatty acids, usually obtained by reducing the fatty acid esters. Likewise, according to the invention, those fatty alcohols can be added that are obtained by reducing naturally occurring triglycerides like beef tallow, palm oil, peanut oil, oil of rapeseed, cotton seed oil, soja oil, sunflower oil and linen oil, or the fatty acid esters produced from their transesterification products with appropriate alcohols, thereby producing a mixture of different fatty alcohols. Such substances can be bought, for example, under the trade names Stenol®, e.g., Stenol® 1618 or Lanette®, e.g., Lanelte® O or Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Of course, wool wax alcohols such as those that are commercially available, for example, under the trade names Corona®, White Swan®, Coronet® or Fluilan® can also be added according to the invention. The fatty alcohols are added in quantities of 0.1-20 wt. %, based on the total preparation, preferably in quantities of 0.1-10 wt. %.

According to the invention, solid paraffins or isoparaffins, carnauba wax, bean wax, candelilla wax, ozocerite, ceresine, sperm wax, sunflowerwax, fruit waxes such as, for example, apple wax or citrus wax, microwaxes from PE or PP can be added as the natural waxes. These types of waxes are available, for example, from Kahl & Co., Trittau.

Exemplary natural and synthetic cosmetic oil bodies, which can augment the action of the inventive active principle, include:
vegetal oils. Examples of such oils are sunflower oil, olive oil, soya oil, rapeseed oil, almond oil, jojoba oil, orange oil, wheat germ oil, peach stone oil and the liquid parts of coconut oil. Other triglyceride oils such as the liquid fractions of beef tallow as well as synthetic triglyceride oils are also suitable, however.
liquid paraffin oils, isoparaffin oils and synthetic hydrocarbons as well as di-n-alkyl ethers containing a total of 12 to 36 carbon atoms, particularly 12 to 24 carbon atoms such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.butyl ether, diisopentyl ether, di-3-ethyldecyl ether, tert.butyl n-octyl ether, isopentyl n-octyl ether and 2-methylpentyl n-octyl ether. The commercial products 1,3-di-(2-ethylhexyl)cyclohexane (Cetiol® S) and di-n-octyl ether (Cetiol® OE) can be preferred.
Ester oils. Ester oils are understood to mean the esters of C₆-C₃₀ fatty acids with C₂-C₃₀ fatty alcohols. Monoesters of fatty acids with alcohols having 2 to 24 carbon atoms are preferred. Examples of added fatty acids moieties in the esters are caproiacid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linolic acid, linolenic acid, elaeostearic acid, arachidonic acid, gadoleic acid, behenic acid and erucic acid as well as their technical mixtures, that e.g., result from cracking of natural fats and oils, from the oxidation of aldehydes from Roelen's oxo synthesis or the dimerization of unsaturated fatty acids. Example for the fatty alcohol moieties in the ester oils are isopropyl alcohol, capron alcohol, capryl alcohol, 2-ethylhexyl alcohol, caprin alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol as well as their technical mixtures, that e.g., result from the high pressure hydrogenation of industrial methyl esters based on fats and oils or aldehydes from Roelen's oxo synthesis as well as the monomer fraction on the dimerization of unsaturated fatty alcohols. According to the invention, isopropyl myristate (Rilanit® IPM), isononanoic acid-C16-18-alkyl ester (Cetiol® SN), 2-ethylhexyl palmitate (Cegesoft® 24), Stearic acid 2-ethylhexyl ester (Cetiol® 868), Cetyl oleate, glycerin tricaprylate, cocofatty alcohol caprinate/-caprylate (Cetiol® LC), n-butyl stearate, oleyl erucate (Cetiol®J600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.
Dicarboxylic acid esters such as di-n-butyl adipate, di-(2-ethylhexyl) adipate, di-(2-ethylhexyl) succinate and di-isotridecyl acetate as well as diol esters such as ethylene glycol dioleate, ethylene glycol di-isotridecanoate, propylene glycol di(2-ethylhexanoate), propylene glycol di-isostearate, propylene glycol di-pelargonate, butanediol di-isostearate, neopentyl glycol dicaprylate,
symmetrical, unsymmetrical or cyclic esters of carbon dioxide with fatty alcohols, e.g., described in DE-OS 197 56 454, glycerin carbonate ordicaprylyl carbonate (Cetiol® CC),
mono, di and trifatty acid esters of saturated and/or unsaturated linear and/or branched fatty acids with glycerin, e.g., Monomuls® 90-O18, Monomuls®) 90-L12 or Cutina® MD.

The added quantities are 0.01 to 50 wt. %, based on the total composition, preferably 0.1 to 10 wt. % and particularly preferably 0.1 to 5 wt. %, based on the total composition.

In a preferred embodiment of the invention, the total quantity of the oil and fat components in the inventive agents ranges from 0.01-5 wt. %, based on the total composition. Quantities of 0.1 to 5 wt. % are preferred.

According to a preferred embodiment, the shampoos can further comprise polyols.

Polyols, which are considered in the context of the invention, possess preferably 2 to 15 carbon atoms and at least two hydroxyl groups. Typical examples are
glycerin;
alkylene glycols, such as, for example, ethylene glycol, diethylene glycol, propylene glycol, butylene glycol, hexylene glycol as well as polyethylene glycols with an average molecular weight of 100 to 1000 daltons;
industrial oligoglycerin mixtures with a degree of self condensation of 1.5 to 10 like for instance industrial diglycerin mixtures with a diglycerin content of 40 to 50 wt. %;
methylol compounds like in particular trimethylol ethane, trimethylol propane, trimethylol butane, pentaerythreitol and dipentaerythritol;
lower alkyl glucosides, particularly those with 1 to 8 carbon atoms in the alkyl group, such as, for example, methyl and butyl glucoside;
sugar alcohols with 5 to 12 carbon atoms, such as, for example, sorbitol or mannitol,
sugars with 5 to 12 carbon atoms, such as, for example, glucose or saccharose;
amino sugars, such as, for example, glucamine.

According to the invention, compositions with a mixture of a plurality of polyols are preferred. A mixture of glycerin, sorbitol, 1,2-propylene glycol and polyethylene glycol is particularly preferred.

The polyol mixture is preferably added in the shampoos in quantities of 0.01 to 35 wt. %, an addition quantity in the range 1 to 20 wt. % is particularly preferred.

In addition, the inventive agents can comprise protein hydrolyzates and/or amino acids and their derivatives (H). Protein hydrolyzates are product mixtures obtained by acid-, base- or enzyme-catalyzed degradation of proteins (albumins). According to the invention, the term “protein hydrolyzates” is also understood to mean total hydrolyzates as well as individual amino acids and their derivatives as well as mixtures of different amino acids. Furthermore, according to the invention, polymers built up from amino acids and amino acid derivatives are understood to be included in the term protein hydrolyzates. The latter include, for example, polyalanine, polyasparagine, polyserine etc. Additional examples of usable compounds according to the invention are L-alanyl-L-proline, polyglycine, glycyl-L-glutamine or D/L-methionine-S-methyl sulfonium chloride. Of course, β-amino acids and their derivatives like β-alanine, anthranilic acid or hippuric acid can also be added according to the invention. The molecular weight of the inventive usable protein hydrolyzates ranges between 75, the molecular weight of glycine, and 200 000, preferably the molecular weight is 75 to 50 000 and quite particularly preferably 75 to 20 000 daltons.

According to the invention, the added protein hydrolyzates can be of both vegetal as well as animal or marine or synthetic origin.

Animal protein hydrolyzates are, for example, elastin, collagen, keratin, milk protein, and silk protein hydrolyzates, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol®) (Croda).

According to the invention, it is preferred to use protein hydrolyzates of vegetal origin, e.g., soya-, almond-, pea-, potato- and wheat protein hydrolyzates. Such products are available, for example, under the trade names Gluadin® (Cognis), DiaMin® (Diamalt), Lexein® (Inolex) Hydrosoy® (Croda), Hydrolupin® (Croda), Hydrosesame® (Croda), Hydrotritium® (Croda) and Crotein® (Croda).

Although it is preferred to add the protein hydrolyzates as such, optionally other mixtures containing amino acids can also be added in their place Likewise, it is possible to add derivatives of protein hydrolyzates, e.g., in the form of their fatty acid condensation products. Such products are marketed, for example, under the trade names Lamepon® (Cognis), Lexein® (Inolex), Crolastin® (Croda) or Crotein® ^{â}Croda).

The compositions used according to the invention preferably comprise the protein hydrolyzates or their derivatives in quantities of 0.1 to 10 wt. %, based on the total composition. Quantities of 0.1 to 5 wt. % are particularly preferred.

The use of vitamins, provitamins and vitamin precursors as well as their derivatives in the inventive agents has also proven to be advantageous. According to the invention, such vitamins, provitamins and vitamin precursors are preferred, which are normally classified in the groups A, B, C, E, F and H.

In the group of substances designated as vitamin A, belong retinol (vitamin A₁) as well as 3,4-didehydroretinol, (vitamin A₂). β-carotene is the provitamin of retinol. Examples of suitable vitamin A components according to the invention are vitamin A acid and its esters, vitamin A aldehyde and vitamin A alcohol as well as its esters such as the palmitate and acetate. The preparations used according to the invention preferably comprise the vitamin A components in amounts of 0.05-1 wt. % based on the total preparation.

The vitamin B group or the vitamin B complex include among other things

Vitamin B₁ (Thiamin)

Vitamin B₂ (Riboflavin)

Vitamin B₃.

The compounds nicotinic acid and nicotinamide (niacinamide) are often included under this designation. According to the invention, nicotinamide is preferred and is comprised in the inventively used compositions in amounts of 0.05 to 1 wt. % based on the total composition.

Vitamin B₅ (pantothenic acid, panthenol and pantolactone). In the context of this group, panthenol and/or pantolactone is preferably used. Usable derivatives of panthenol according to the invention are especially the esters and ethers of panthenol as well as cationic derivatized panthenols. Specific representatives are, for example, panthenol triacetate, panthenol monoethyl ether and its monoacetate as well as the cationic panthenol derivatives disclosed in WO 92/13829. The cited compounds of the vitamin B₅ type are comprised in the compositions used according to the invention in amounts of 0.05-10 wt. %, based on the total composition. Quantities of 0.1 to 5 wt. % are particularly preferred. Vitamin B₆ (pyridoxine as well as pyridoxamine and pyridoxal).

Vitamin C (ascorbic acid). Vitamin C is preferably added to the compositions used according to the invention in amounts of 0.1 to 3 wt. %, based on the total composition. Its use in the form of the palmitate ester, the glucosides or phosphates can be preferred. Its use in combination with tocopherols can also be preferred.

Vitamin E (Tocopherols, especially α-tocopherol). Tocopherol and its derivatives, including particularly the esters, such as the acetate, the nicotinate, the phosphate and the succinate, are used in the compositions according to the invention preferably comprised in amounts of 0.05-1 wt. %, based on the total composition.

Vitamin F. The term “vitamin F” is usually taken to mean essential fatty acids, particularly linoleic acid, linolenic acid and arachidonoic acid.

Vitamin H. The compound (3aS,4S,6aR)-2-oxohexahydrothienol[3,4-d]imidazole-4-valeric acid denotes Vitamin H, for which the trivial name biotin has become accepted. The agents used according to the invention preferably comprise biotin in amounts of 0.0001 to 1.0 wt. %, particularly in amounts of 0.001 to 0.01 wt. %.

The preparations according to the invention preferably comprise vitamins, provitamins and vitamin precursors from groups A, B, E and H. Naturally, a plurality of vitamins and vitamin precursors may also be comprised at the same time.

Pantolactone, pyridoxine and its derivatives as well as nicotinamide and biotin are especially preferred.

The added quantity of the vitamins and vitamin precursors in the inventive agent is 0.0001 to 10 wt. %, based on the total composition, preferably 0.0001 to 5 wt. % and particularly 0.0001 to 3 wt. %.

Finally, plant extracts can be used in the inventive agents. Usually, these extracts are manufactured by extraction of the whole plant. In individual cases, however, it can also be preferred to manufacture the extracts solely from blossoms and/or leaves of the plant.

] With regard to the inventively usable plant extract, we particularly refer to extracts that are listed in the Table beginning on page 44 of the 3rd edition of the Guidelines for the Declaration of Ingredients in Cosmetics, (Leiffadens zur Inhaltsstoffdeklaration kosmetischer Mittel) published by the German Cosmetics, Toiletry, Perfumery and Detergent Association e.V. (IKW), Frankfurt.

According to the invention, mainly extracts from green tea, oak bark, stinging nettle, hamamelis, hops, henna, chamomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althaea), meristem, ginseng and ginger are preferred.

Extracts from green tea, oak bark, stinging nettle, hamamelis, hops, chamomile, burdock root, hawthorn, linden flowers, almonds, aloe vera, coconut, mango, apricot, lime, wheat, kiwi, melon, orange, grapefruit, sage, rosemary, birch, lady's smock, common yarrow, rest-harrow, meristem, ginseng and ginger are preferred.

Extracts of green tea, almonds, aloe vera, coconut, mango, apricot, lime, wheat, kiwi and melon are quite particularly suitable for the inventive use.

The extracting agent used to prepare the cited plant extracts can be water, alcohols as well as their mixtures. Exemplary preferred alcohols are lower alcohols such as ethanol and isopropanol, but particularly polyhydroxy alcohols such as ethylene glycol, propylene glycol and butylene glycol, both as the sole extracting agent as well as in aqueous mixtures. Plant extracts based on water/propylene glycol in the ratio 1:10 to 10:1 have proven particularly suitable.

According to the invention, the plant extracts can be used in pure and also in diluted form. When they are used in diluted form, they normally comprise ca. 2-80 wt. % active substance and the solvent is the extracting agent or mixture of extracting agents used for their preparation.

In addition, it can be preferred to add mixtures of a plurality, particularly two different plant extracts to the inventive agent.

The added quantity of the plant extracts in the inventive agent is 0.01 to 50 wt. %, based on the total composition, preferably 0.1 to 30 wt. % and particularly 0.1 to 20 wt. %.

In the context of the invention, preferred cationic polymers are, for example, cationic cellulose derivatives, such as e.g., a quaternized hydroxyethyl cellulose, available under the trade name Polymer JR®400 from Amerchol, cationic starches, copolymers of diallylammonium salts and acrylamides, quaternized vinyl pyrrolidone/vinyl imidazole polymers, such as e.g., Luviquat® (BASF), condensation products of polyglycols with amines, quaternized collagen polypeptides, such as, for example, lauryldimonium hydroxypropyl hydrolyzed collagen (Lamequat®L/Grünau), quaternized wheat polypeptides, polyethylene imine, cationic silicone polymers, such as amidomethicone, copolymers of adipic acid and dimethylamino hydroxypropyl diethylene triamine (Cartaretine®/Sandoz), copolymers of acrylamide and dimethyldiallylammonium chloride (Merquat® 550/Chemviron), homopolymers of dimethyldiallylammonium chloride (Merquate 100), polyaminopolyamides, such as e.g., described in FR A 2252840 as well as their crosslinked water-soluble polymers, cationic chitin derivatives such as e.g., quaternized chitosan, optionally microcrystallinically dispersed, condensation products of dihaloalkylenes, such as e.g., dibromobutane with bisdialkylamines, such as e.g., bis-dimethylamino-1,3-propane, cationic guar gum, such as, for example, Jaguar® CBS, Jaguar® C-1 7, Jaguar® C-16 from Celanese, quaternized ammonium salt polymers such as e.g., Mirapol® A-15, Mirapol® AD-1, Mirapol® AZ-1 from Miranol.

Particularly preferred cationic polymers are cationic guar derivatives, cationic cellulose derivatives, homopolymers of dimethyldiallylammonium chloride as well as copolymers of dimethyldiallylammonium chloride with acrylamide.

Suitable nonionogenic polymers are, for example:
vinyl pyrrolidone-vinyl ester copolymers, such as, for example, those marketed by BASF under the trade name Luviskol®, Luviskol® VA 64 and Luviskol® VA 73, each vinyl pyrrolidone-vinyl acetate copolymers, are likewise preferred nonionic polymers.
Cellulose ethers, such as hydroxypropyl cellulose, hydroxyethyl cellulose, and methyl hydroxypropyl cellulose, as marketed, for example, under the trademarks Culminal® and Benecel® (AQUALON).
shellac
polyvinyl pyrrolidones, as are marketed, for example, under the designation Luviskol® (BASF).
siloxanes. These siloxanes can be both water-soluble and water-insoluble. Both volatile and non-volatile siloxanes are suitable, whereby non-volatile siloxanes are understood to mean such compounds with a boiling point above 200° C. at normal pressure. Preferred siloxanes are polydialkylsiloxanes, such as, for example, polydimethylsiloxane, polyalkylarylsiloxanes, such as, for example, polyphenylmethylsiloxane, ethoxylated polydialkylsiloxanes as well as polydialkylsiloxanes, which comprise amine and/or hydroxyl groups.
glycosidically substituted silicones according to EP 0612759 B1.

The compositions used according to the invention preferably comprise the polymers in quantities of 0.01 to 10 wt. %, based on the total composition. Quantities of 0.1 to 5 wt. %, particularly 0.1 to 3 wt. %, are particularly preferred.

In the context of the invention it can, in addition, be advantageous to add short chain carboxylic acids. In the context of the invention, short chain carboxylic acids and their derivatives are understood to mean carboxylic acids that can be saturated or unsaturated and/or linear or branched or cyclic and/or aromatic and/or heterocyclic and have a molecular weight of less than 750. In the context of the invention, saturated or unsaturated or linear or branched carboxylic acids with a chain length of 1 to 16 carbon atoms in the chain can be preferred, those with a chain length of 1 up to 12 carbon atoms in the chain are quite particularly preferred.

In the context of the invention, the short chain carboxylic acids can have one, two, three or more carboxyl groups. In the context of the invention, carboxylic acids with a plurality of carboxyl groups are preferred, particularly di and tricarboxylic acids. The carboxyl groups can be totally or partially present as esters, acid anhydrides, lactones, amides, imide acid, lactams, lactims, dicarboximides, carbohydrazide, hydrazone, hydroxams, hydroxims, amidines, amidoximes, nitriles, phosphonate- or phosphate esters. The inventive carboxylic acids can of course be substituted along the carbon chain or on the cyclic structure. The substituents of the inventive carboxylic acids include, for example, C1-C8-alkyl-, C2-C8-alkenyl-, aryl-, aralkyl- and aralkenyl-, hydroxymethyl-, C2-C8-hydroxyalkyl-,C2-C8-hydroxyalkenyl-, aminomethyl-, C2-C8-aminoalkyl-, cyano-, formyl-, oxo-, thioxo-, hydroxy-, mercapto-, amino-, carboxyl- or imino groups. Preferred substituents are C1-C8-alkyl-, hydroxymethyl-, hydroxy-, amino- and carboxyl groups. Substituents in the α-position are particularly preferred. Quite particularly preferred substituents are hydroxy-, alkoxy- and amino groups, wherein the amino function can be optionally further substituted by alkyl, aryl, aralkyl and/or alkenyl groups. In addition, equally preferred carboxylic acid derivatives are the phosphonate- and phosphate esters.

Exemplary inventive carboxylic acids are formic acid acetic acid, propionic acid, butyric acid, isobutyric acid, valerianic acid, isovalerianic acid, pivalic acid, oxalic acid, malonic acid, succinic acid, glutaric acid, glycerinic acid, glyoxylic acid, adipic acid, pimelic acid, cork acid, azelaic acid, sebacic acid, propiolic acid, crotonic acid, isocrotonic acid, elaidic acid, maleic acid, fumaric acid, muconic acid, citraconic acid, mesaconic acid, campheric acid, benzoic acid, o,m,p-phthalic acid, naphthoic acid, toluoylic acid, hydratropic acid, atropic acid, cinnamic acid, isonicotinic acid, nicotinic acid, bicarbaminic acid, 4,4′-dicyano-6,6′-binicotinic acid, 8-carbamoyloctanoic acid, 1,2,4-pentanetricarboxylic acid, 2-pyrrolcarboxylic acid, 1,2,4,6,7-napthalinpentaacetic acid, malonaldehydic acid, 4-hydroxy-phthalamidic acid, 1-pyrazolcarboxylic acid, gallic acid or propanetricarboxylic acid, a dicarboxylic acid selected from the group made up of compounds of the general formula (N—I),
(N—I)
in which Z stands for a linear or branched alkyl or alkenyl radical containing 4 to 12 carbon atoms, n for a number from 4 to 12 as well as one of both the groups X and Y for a COOH group and the other for hydrogen or a methyl or ethyl group, dicarboxylic acids of the general formula (N—I), which additionally have 1 to 3 methyl or ethyl substituents on the cyclohexene ring as well as dicarboxylic acids that are obtained from the dicarboxylic acids according to formula (N—I) by the formal addition of a molecule of water on the double bond in the cyclohexene ring.

Dicarboxylic acids of Formula (N—I) are known in the literature.

A production process is known, for example, from U.S. Pat. No. 3,753,968.

The dicarboxylic acids of Formula (N—I) can be manufactured, for example, by a Diels-Alder cyclization by reacting polyunsaturated carboxylic acids with unsaturated monocarboxylic acids. Usually, a polyunsaturated fatty acid is the starting material for the dicarboxylic acid component. Linoleic acid obtained from natural fats and oils is preferred. Acrylic acid, but also e.g., methacrylic acid and crotonic acid are particularly preferred as the monocarboxylic acid. Diels-Alder reactions usually result in mixtures of isomers, in which one component is in excess. According to the invention, this mixture of isomers can be added just like the pure compound.

According to the invention, besides the preferred dicarboxylic acids according to Formula (N—I), other such dicarboxylic acids can be added; they differ from the compounds of Formula (N—I) by the 1 to 3 methyl or ethyl substituents on the cyclohexene ring or are formed from these compounds by the formal addition of one molecule water on the double bond of the cyclohexene ring.

The dicarboxylic acid (mixture) from the reaction of linoleic acid with acrylic acid has proven to be particularly inventively advantageous. It is a mixture of 5- and 6-carboxy-4-hexyl-2-cyclohexene-1-octanoic acid. Such compounds are commercially obtainable under the trade names Westvaco Diacid® 1550 and Westvaco Diacid® 1595 (Manufacturer: Westvaco).

Besides the exemplary previously listed short chain carboxylic acids themselves, their physiologically acceptable salts can also be added according to the invention. Examples of such salts are the alkali- alkaline earth-, zinc salts as well as ammonium salts, under which in the context of the present invention are also understood to mean the mono-, di- and trimethyl-, -ethyl and -hydroxyethyl ammonium salts. However, with alkaline reacting amino acids such as, for example, arginine, lysine, ornithine and histidine, in the context of the invention, it is quite particularly preferred to be able to add neutralized acids. Moreover, from formulation grounds, it can be preferred to select the carboxylic acid from the water-soluble representatives, in particular the water-soluble salts.

In addition, it is inventively preferred to add hydroxycarboxylic acids and here once again the dihydroxy-, trihydroxy- and polyhydroxy carboxylic acids as well as the dihydroxy-, trihydroxy- and polyhydroxy di-, tri- and polycarboxylic acids. In this respect, it was shown that besides the hydroxycarboxylic acids, also the hydroxycarboxylic acid esters as well as mixtures of hydroxycarboxylic acids and their esters and also polymeric hydroxycarboxylic acids and their esters can be quite particularly preferred. Preferred hydroxycarboxylic acid esters are fully esterified glycolic acid, lactic acid, malic acid, tartaric acid or citric acid, for example. Additional fundamentally suitable hydroxycarboxylic acid esters are esters of β-hydroxypropionic acid, of tartronic acid, of D-gluconic acid, of saccharic acid, of mucic acid or of glucuronic acid. Primary, linear or branched aliphatic alcohols with 8-22 carbon atoms, i.e., fatty alcohols or synthetic fatty alcohols are suitable alcohol moieties of these esters. Esters of C12-C15 fatty alcohols are particularly preferred in this respect. Esters of this type are commercially available, e.g., under the trade name Cosmacol® from Enichem, Augusta Industriale. Particularly preferred polyhydroxypolycarboxylic acids are polylactic acid and polytartaric acid as well as their esters.

According to a preferred embodiment, the inventive compositions can further comprise anionic polymers.

The homopolymer of 2-acrylamido-2-methylpropane sulfonic acid, which is commercially available, for example, under the trade name Rheothik® 11-80, is particularly preferred.

In this embodiment, it can be preferred to use copolymers of at least one anionic monomer and at least one nonionic monomer. Regarding the anionic monomers, reference is made to the above-mentioned substances. Preferred non-ionic monomers are acrylamide, methacrylamide, acrylic acid esters, methacrylic acid esters, vinyl pyrrolidone, vinyl ethers and vinyl esters.

Preferred anionic copolymers are acrylic acid-acrylamide copolymers and particularly polyacrylamide copolymers with monomers that contain sulfonic acid groups. A particularly preferred anionic copolymer consists of 70 to 55 mole % acrylamide and 30 to 45 mole % 2-acrylamido-2-methylpropane sulfonic acid, wherein the sulfonic acid group may be fully or partially present as the sodium, potassium, ammonium, mono ortriethanolammonium salt. This copolymer can also be crosslinked, wherein the preferred crosslinking agents include polyolefinic unsaturated compounds such as tetraallyloxyethane, allylsucrose, allylpentaerythritol and methylene bisacrylamide. Such a polymer is comprised in the commercial product Sepigel® 305 from the SEPPIC company. The use of this compound, which comprises a mixture of hydrocarbons (C₁₃-C₁₄ isoparaffin) and a nonionic emulsifier (Laureth-7) besides the polymer components, has proved to be particularly advantageous in the context of the inventive teaching.

The sodium acryloyl dimethyl taurate copolymers commercialized as a compound with isohexadecane and polysorbate 80, under the trade name Simulgel® 600, have also proved to be particularly effective according to the invention.

Likewise preferred anionic homopolymers are uncrosslinked and crosslinked polyacrylic acids. Here the preferred crosslinking agents can be allyl ethers of pentaerythritol, of sucrose and of propylene. Such compounds are commercially available under the trade name Carbopol®, for example.

Copolymers of maleic anhydride and methyl vinyl ether, especially those with crosslinks are also color-conserving polymers. A maleic acid-methyl vinyl ether copolymer, crosslinked with 1,9-decadiene is commercially available under the name Stabileze® QM.

Exemplary additional active substances, adjuvants and additives, which can be added in minor quantities, are
additional thickeners like agar-agar, guar gum, alginates, xanthane gum, gum arabica, karaya gum, locust bean flour, linseed gums, dextrans, cellulose derivatives, e.g., methyl cellulose, hydroxyalkyl cellulose and carboxymethyl cellulose, starch fractions and derivatives like amylose, amylopectin and dextrins, clays such as e.g., bentonite or synthetic hydrocolloids such as e.g., polyvinyl alcohol,
hair conditioning compounds like phospholipids, for example, soya lecithin, egg lecithin and cephalin, as well as silicone oils,
perfume oils, dimethyl isosorbitol and cyclodextrins,
solvents and solubilizers like ethanol, isopropanol, ethylene glycol, propylene glycol, glycerin and diethylene glycol,
fiber structure improvers, particularly mono, di and oligosaccharides, such as, for example, glucose, galactose, fructose, fruit sugar and lactose,
conditioning active substances like paraffin oils, vegetal oils, e.g., sunflower oil, orange oil, almond oil, wheat germ oil, peach stone oil as well as quaternized amines like methyl-1-alkylamidoethyl-2-alkyl imidazolinium methosulfate,
defoamers like silicones,
dyestuffs to color the composition,
anti-dandruff active materials like piroctone olamine, zinc omadine and climbazole,
Active principles like allantoin and bisabolol, cholesterine,
thickeners like sugar esters, polyol esters or polyol alkyl ethers,
fats and waxes like spermaceti, beeswax, montan wax and paraffins,
fatty acid alkanolamides,
chelating agents like EDTA, NTA, β-alanine diacetic acid and phosphonic acids,
swelling and penetration agents like primary, secondary and tertiary phosphates,
opacifiers like latex, styrene/PVP copolymers and styrene/acrylamide copolymers,
pearlizing agents like ethylene glycol mono and distearate as well as PEG-3-distearate,
pigments,
blowing agents like propane-butane mixtures, N₂O, dimethyl ether, CO₂ and air,
antioxidants,
pH regulators such as, for example, citric acid or lactic acid,
preservatives such as, for example, benzoic acid or salicylic acid.

With regard to further optional ingredients and their amounts used, reference is expressly made to the relevant handbooks known to the expert, for example, the monograph by K. Schrader, Grundlagen and Rezepturen der Kosmetika, 2nd edition, Hüthig Buch Verlag, Heidelberg, 1989.

A second subject of the invention is the use of an essentially anionic surfactant-free cleaning agent with a content of 1 to 50 wt. % of a cationic surfactant of Formula (I) 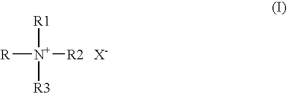
wherein
R stands for an alkyl group with 8 to 18 carbon atoms, preferably for an alkyl group with 8 to 16 carbon atoms and particularly for an alkyl group with 10 to 14 carbon atoms.

R¹, R², R³ stand independently of one another for a C₁-C₃-alkyl group and

X⁻ stands for halogen or methosulfate, for conditioning hair.

A third subject of the invention is the use of the inventive cleaning agent to protect the hair color.

A fourth subject of the invention is the use of the inventive cleaning agent for reducing and preventing damage to hair.

### EXAMPLES

The following shampoos were prepared in the context of the invention. The quantities refer to wt. %.

| | | |
|---|---|---|
| 1) | Cocotrimonium Methosulfate (30% AS) | 35.0 |
| | Cocamide MEA | 2.5 |
| | Cocamidopropyl Betaine (30% AS) | 5.0 |
| | Hydroxypropyl Guar (Jaguar ® HP-200) | 1.0 |
| | Polyethylene imine | 0.3 |
| | Lactic Acid | q.s |
| | Perfume | 0.3 |
| | Water | ad 100 |
| 2) | Cocotrimonium Methosulfate (30% AS) | 35.0 |
| | Cocamide MEA | 2.5 |
| | Cocamidopropyl Betaine (30% AS) | 5.0 |
| | Hydroxypropyl Guar (Jaguar ® HP-40) | 1.0 |
| | Dimethicone (150 000 cSt) | 1.0 |
| | Lactic Acid | q.s |
| | Perfume | 0.3 |
| | Water | ad 100 |
| 3) | Cocotrimonium Methosulfate (30% AS) | 35.0 |
| | Cocamide MEA | 2.5 |
| | Cocamidopropyl Betaine (30% AS) | 5.0 |
| | Hydroxypropyl Guar (Jaguar ® HP-60) | 1.0 |
| | Polyquaternium-55 (20% AS) | 1.0 |
| | Lactic Acid | q.s |
| | Perfume | 0.3 |
| | Water | ad 100 |
| 4) | Cocotrimonium Methosulfate (30% AS) | 35.0 |
| | Cocamide MEA | 2.5 |
| | Cocamidopropyl Betaine (30% AS) | 5.0 |
| | Hydroxypropyl Guar (Jaguar ® HP-60) | 1.0 |
| | Behentrimonium Chloride | 1.0 |
| | Lactic Acid | q.s |
| | Perfume | 0.3 |
| | Water | ad 100 |
| 5) | Cocotrimonium Methosulfate (30% AS) | 35.0 |
| | Cocamide MEA | 2.5 |
| | Cocamidopropyl Betaine (30% AS) | 5.0 |
| | Hydroxypropyl Guar (Jaguar ® HP-120) | 1.0 |
| | Lactic Acid | q.s |
| | Perfume | 0.3 |
| | Water | ad 100 |
| 6) | Cocotrimonium Methosulfate (30% AS) | 35.0 |
| | Cocamide MEA | 2.5 |
| | Cocamidopropyl Betaine (30% AS) | 5.0 |
| | Hydroxypropyl Guar (Jaguar ® HP-60) | 0.5 |
| | Hydroxyethyl Ethylcellulose | 0.5 |
| | Lactic Acid | q.s |
| | Perfume | 0.3 |
| | Water | ad 100 |

1. A cleaning agent for keratinic fibers comprising based on the weight of the cleaning agent from 1 to 50 wt. % of one or more cationic surfactants of Formula (I) 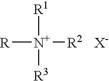
wherein R is an alkyl group having from 8 to 18 carbon atoms; each of R¹, R², and R³ is independently a C₁-C₃-alkyl group and X⁻ is a halogen or methosulfate, wherein the cleaning agent is essentially free of anionic surfactants.

2. The cleaning agent of claim 1, wherein R is an alkyl group having from 8 to 16 carbon atoms.

3. The cleaning agent of claim 2, wherein R is an alkyl group with 10 to 14 carbon atoms.

4. The cleaning agent of claim 1, wherein the cationic surfactant is selected from the group consisting of cocotrimonium chloride, cocotrimonium methosulfate, laurtrimonium chloride, laurtrimonium methosulfate and combinations thereof.

5. The cleaning agent of claim 1, wherein the amount of the cationic surfactant is from 5 to 30 wt. %.

6. The cleaning agent of claim 1, further comprising from 0.1 to 5 wt. % of one or more foam strengtheners selected from the group consisting of the non-ionic surfactants, amphoteric surfactants and combinations thereof.

7. The cleaning agent of claim 6, wherein the foam strengthener is selected from the group consisting of the fatty acid alkanolamides, betaines, amine oxides sugar surfactants and combinations thereof.

8. The cleaning agent of claim 6, wherein the foam strengthener is selected from the group consisting of cocamide MEA, cocamide DEA, cocamide MIPA, PEG-2 cocamide, PPG-2 hydroxyethyl cocamide, PPG-2 hydroxyethyl coco/lsostearamide, alkylamidopropyl betaines, alkylamidopropylamine oxides and combinations thereof.

9. The cleaning agent of claim 1, further comprising from 0.05 to 2 wt. % of a polyalkylene imine.

10. The cleaning agent of claim 9, wherein the amount of the polyalkylene imine is from 0.1 to 1 wt. %

11. The cleaning agent of claim 9, wherein the polyalkylene imine is polyethylene amine.

12. The cleaning agent of claim 1, further comprising from 0.1 to 3 wt. %, of at least one thickener selected from the group consisting of hydroxypropyl guar derivatives, hydroxyethyl ethylcellulose derivatives and combinations thereof.

13. The cleaning agent of claim 13, wherein the amount of the thickener is from 0.3 to 2 wt. %.

14. The cleaning agent of claim 1 comprising from 0.05 to 3 wt. % of an additional cationic surfactant selected from the group consisting of a C₁₆₋₁₈ alkyltrimethylammonium chloride or methosulfate, a C₁₆₋₁₈ dialkyldimethylammonium chloride or methosulfate, a C₁₆₋₁₈ trialkylmethylammonium chloride or methosulfate and combinations thereof.

15. The cleaning agent of claim 14, wherein the amount of the cationic surfactant is from 0.1 to 2 wt. %.

16. The cleaning agent of claim 1, further comprising from 0.05 to 2 wt. % of at least one styling polymer selected from the group consisting of polyquaternium-55, polyquaternium-46, polyquaternium-11, polyquaternium-28 and combinations thereof.

17. The cleaning agent of claim 16, wherein the amount of the styling polymer is from 0.1 to 1 wt. %.

18. The cleaning agent of claim 1, further comprising from 0.05 to 3 wt. % of a water-soluble or water-insoluble, volatile or non-volatile silicone component.

19. A method forconditioning haircomprising contacting hairwith a conditioning effective amount of a cleaning agent comprising based on the weight of the cleaning agent from 1 to 50 wt. % of one or more cationic surfactants of Formula (I) 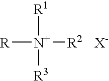
wherein R is an alkyl group having from 8 to 18 carbon atoms; each of R¹, R², and R³ is independently a C₁-C₃-alkyl group and X⁻ is a halogen or methosulfate, wherein the cleaning agent is essentially free of anionic surfactants.
